(19) United States
(12) Patent Application Publication
(10) Pub. No.: US 2009/0125093 A1
(43) Pub. Date: May 14, 2009

(54) Method and apparatus for introducing expandable intraluminal prosthesis

(75) Inventor: Palle M. Hansen, Bjaeverskov (DK)

Correspondence Address: COOK GROUP PATENT OFFICE, P.O. BOX 2269, BLOOMINGTON, IN, 47402, US

(73) Assignees: William Cook Europe ApS, Bjaeverskov (DK); Cook Incorporated, Bloomington (US)

(21) Appl. No.: 12/291,125

(22) Filed: Nov. 6, 2008

Related U.S. Application Data
(60) Provisional application No. 61/002,200, filed on Nov. 7, 2007.

(51) Int. Cl. A61F 2/06; A61B 19/00

(52) U.S. Cl. 623/1.11; 623/1.12; 623/1.23; 128/898

(57) ABSTRACT

A stent introducer has a delivery sheath positioned to restrain a self expandable stent. A pull wire is connected with the delivery sheath and extends through a pull wire lumen to enable remote retraction of the delivery sheath. The distal end of the pull wire lumen is associated with a region of weakness in the structure such that the region of weakness is torn on retraction of the delivery sheath.

### CROSS-REFERENCE TO RELATED APPLICATIONS

This application claims priority of provisional application Ser. No. 61/002,200, filed Nov. 7, 2007.

### TECHNICAL FIELD

The present invention relates to medical devices and in particular to a method and apparatus for introducing an expandable prosthesis into body lumens.

### BACKGROUND OF THE INVENTION

A wide variety of techniques exist for introducing self-expandable prostheses into body lumens. One such technique involves a surgeon advancing a guide-wire through body lumens so that the wire extends through the passage in which it is desired to implant the self-expandable prosthesis, such as a stent. An introducer is then passed over the guide-wire from outside the patient to deliver the stent to the desired location.

A typical introducer comprises a catheter having a wire guide lumen and an atraumatic tip at its distal end. The stent is positioned just proximally of the distal tip of the catheter. An outer sheath extends over the exterior surfaces of the catheter and the stent, so as to restrain the stent during introduction. This introducer is advanced to the deployment location within a body lumen, whereupon the surgeon retracts the outer sheath relative to the catheter so as to gradually unsheathe the self-expanding stent and allow it to engage the vessel wall.

Retraction of an outer sheath which extends the length of the catheter can present problems. The length of sheath that has to be moved and the frictional resistance to movement can lead to uncertainties in the positioning of the stent. There can also be an increased risk of damage to the intimal cells. For this reason, it has been proposed in prior art constructions to reduce the length of the outer sheath; a shortened outer sheath is located near the distal end of the introducer covering only the stent and—usually—a small portion of the distal end of the catheter. Typically, a pull wire is attached to the shortened outer sheath to enable it to be retracted remotely.

If a pull wire becomes twisted circumferentially around the catheter, there is not only an increased frictional resistance, but also the risk of unwanted torque being applied to the outer sheath. It has for this reason been suggested to locate the pull wire in a lumen within the catheter. It is necessary, however, to accommodate retraction of the sheath and for this reason the pull wire lumen is replace by on open channel or groove at the distal end of the catheter, over a length at least equal to the length of the outer sheath.

There remain difficulties with such an arrangement. That portion of the pull wire that lies within the open channel or groove can become dislodged and prone to twisting. This risk will increase with the length of the channel or groove and it is therefore desirable for the length of the channel or groove to match that of the sheath. This however creates manufacturing and inventory problems since a different catheter must be designed for each sheath length.

### SUMMARY OF THE INVENTION

Thus, there is provided in accordance with one aspect of the present invention apparatus for delivering an expandable prosthesis within a body lumen, comprising a catheter structure having at its distal end a prosthesis bearing site; a delivery sheath positioned over said prosthesis bearing site so as to retain a prosthesis as the catheter structure is advanced through the body lumen, the delivery sheath being retractable to release the prosthesis; a pull wire lumen extending at least part way through the catheter structure from the proximal end of the delivery sheath; and a pull wire connected with the delivery sheath and extending through the pull wire lumen to enable remote retraction of the delivery sheath, wherein the distal end of the pull wire lumen is associated with a region of weakness in the catheter structure such that the region of weakness is torn on retraction of the delivery sheath.

The pull wire lumen can extend directly to the sheath with no need for an open channel or groove to accommodate retraction of the sheath. During introduction of the prosthesis to the delivery site, the entire length of the or each pull wire can accordingly remain within the corresponding pull wire lumen, with no risk of circumferential twisting. One catheter may be designed for combination with a range of delivery sheaths of varying lengths, the different retracting lengths being accommodated by tearing of different lengths of the region of weakness associated with the pull wire lumen.

The exterior surface of the catheter structure, free of grooves, may assist in advancement of the catheter structure. The reaction of the catheter structure to initial proximal retraction of the delivery sheath—before sufficient force is applied to tear the region of weakness—may assist in resisting early or inadvertent retraction of the delivery sheath.

The catheter structure may comprise an inner sheath, said pull wire lumen and said associated region of weakness being located in the inner sheath. The apparatus may further comprise a barb that, on retraction of the delivery sheath, aids in tearing said region of weakness, the barb being formed on an annular member located around said catheter structure or on the delivery sheath. There may be a plurality of pull wires and optionally also of such barbs spaced equiangularly about said catheter structure.

The regions of weakness may be provided at least in part by grooves formed in said catheter structure. An inner sheath may consist substantially of a first material with the regions of weakness being provided at least in part by elongate regions of a second material having optionally greater shear strength or greater scratch hardness than said first material. The second material may be anisotropic.

According to a further aspect of the invention, there is provided a method for delivering an expandable prosthesis within body lumens, said method comprising the steps of positioning in a body lumen an apparatus having a longitudinal axis, said apparatus comprising a catheter structure having at its distal end a prosthesis bearing site; an expandable prosthesis located at said prosthesis bearing site; a delivery sheath positioned over said prosthesis bearing site so as to retain the prosthesis as the catheter structure is advanced through the body lumen, the delivery sheath being retractable to release the prosthesis; a pull wire lumen extending at least part way through the catheter structure from the proximal end of the delivery sheath; and a pull wire connected with the delivery sheath and extending through the pull wire lumen to enable remote retraction of the delivery sheath, wherein the distal end of the pull wire lumen is associated with a region of weakness in the catheter structure; and pulling said pull wire proximally along said axis so as to retract said delivery sheath, thus unsheathing said prosthesis, and tearing said region of weakness.

Further objects, features and advantages of this invention will become readily apparent to person skilled in the art after a review of the following description, with reference to the drawings and claims that are appended to and form a part of this specification.

### BRIEF DESCRIPTION OF THE DRAWING

FIG. 1 is an axial section through the distal region of an introducer in accordance with a first embodiment of the present invention

FIG. 2 is a partial isometric view of the distal end of the inner sheath of the first embodiment before retraction of the outer delivery sheath.

FIG. 3 is a view similar to that of FIG. 2 following partial retraction of the outer sheath.

FIG. 4 is a radial cross sectional view of an introducer according to a further embodiment of the present invention.

FIG. 5 is a radial cross sectional view of an introducer according to a still further embodiment of the present invention.

FIG. 6 is a radial cross sectional view of yet a further embodiment of the present invention having an annular cutting member.

FIG. 7 is an axial cross sectional view of the introducer depicted in FIG. 1 within a body lumen before deployment of a self-expanding stent.

FIG. 8 is a view similar to that of FIG. 7 with the self-expanding stent partially deployed.

FIG. 9 is a view similar to that of FIGS. 7 and 8 with the self-expanding stent fully deployed.

### DETAILED DESCRIPTION

The example will be taken of apparatus for delivering an expandable prosthesis within body lumens in the form of an introducer for delivering a self-expandable stent.

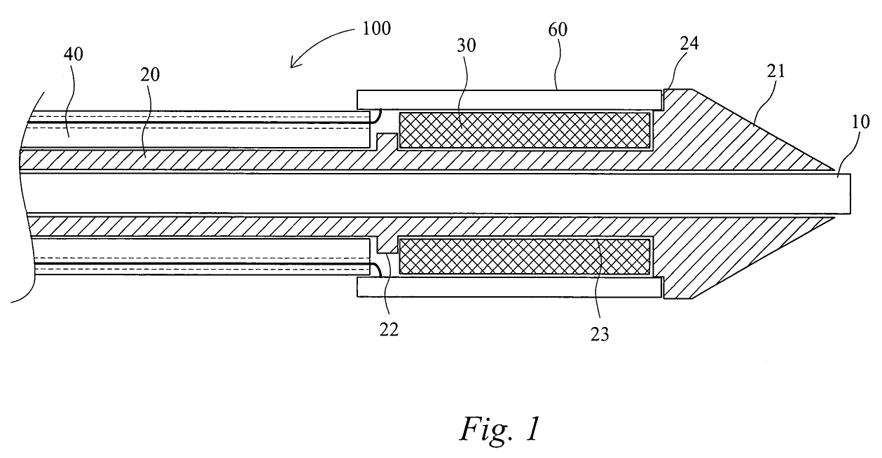

FIG. 1 shows an introducer (100) comprising a catheter (20) configured for introduction over a guide wire (10). The catheter (20) provides at its distal end a bearing site (23) for a stent (30), the bearing site being defined between an atraumatic distal tip (21) and a stent pusher ring (22). The atraumatic distal tip (21) and the stent pusher ring (22) may be formed integrally in the catheter (20) or may—through a variety of techniques well known in the art—be formed from separate components bonded or appropriately secured to the core catheter. The atraumatic distal tip (21) may have a substantially frustoconical shape.

An inner sheath (40) extends coaxially over the catheter (20) from the proximal end of the catheter to the stent pusher ring (22). This inner sheath (40) will typically be of a different material than that of the catheter (20) and may be appropriately bonded or secured to the catheter so as to be fixed against movement relative to the catheter (20) whether axial or circumferential. An outer or delivery sheath (60), which is very much shorter than the catheter (20) and the inner sheath (40) and which may be only just longer than the stent (30), is disposed over the stent (30) preferably covering the distal extremity of the inner sheath (40). The distal tip (21) of the catheter is preferably provided with an annular recess 23 to accommodate the distal end of the outer sheath. As will subsequently be described in more detail, the outer or delivery sheath (60) is retractable proximally relative to the catheter to release the stent.

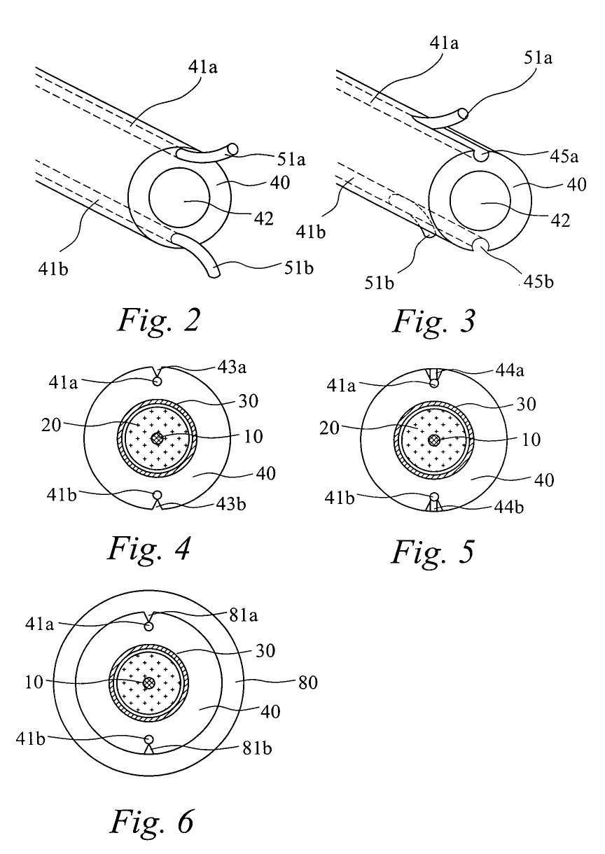

The inner sheath (40) will now be described in more detail, making reference also to FIGS. 2 and 3.

The inner sheath (40) has a main lumen (42) which extends along its longitudinal axis so as to make the inner sheath (40) substantially tubular. The inner sheath also comprises two pulling wire lumens (41a, 41b) which are disposed either side of the central lumen (42) and extend parallel to the longitudinal axis. These are equiangularly spaced about the longitudinal axis. Disposed within each pulling wire lumen (41a, 41b) is one of two pulling wires (50a, 50b) which extend along the length of the introducer and may be operated by the surgeon from exterior the patient. The pulling wires (50a, 50b) emerge from the distal end of the inner sheath through the distal ends of the pulling wire lumens (41a, 41b) and are attached to the interior of the outer sheath (60). The inner sheath (40) may comprise any suitable biocompatible material such as polytetrafluoroethylene (PFTE), polyetheretherketones (PEEK), polyvinyl chloride (PVC), polyimide, polyimide reinforced with a stainless steel braid (provided interior the pulling wire lumens 41a, 41b), polyurethane and the like. The exterior of inner sheath (40) may be provided with a lubricous coating so as to reduce friction between the outer surface and the body lumens during introduction and to reduce friction between the outer sheath and inner sheath during retraction of the outer sheath. It will be noted that in this configuration, the exterior surface of the inner sheath is smooth and continuous.

FIG. 3 displays the inner sheath (40), with the pulling wires (50a, 50b) partially retracted so as to pull back the outer sheath (not shown in FIG. 3) and partially unsheathe the stent (not shown in FIG. 3) which is located adjacent the distal end of the inner sheath (40). The pulling wire lumens (41a, 41b) are situated sufficiently close to the surface of inner sheath (40) that when the pulling wires (50a, 50b) are retracted the distal ends of the pulling wires (51a, 51b) tear slits (45a, 45b) in the radially exterior surface of the inner sheath (40). Thus, the outer sheath (60) may only be retracted by tearing of the material of inner sheath (40) and specifically by the tearing of a region of weakness comprising a strip region of the inner sheath bounding the associated pull wire lumen to the radially outward side. The weakness arises in this case from the small radial dimension of the strip region and from the material chosen for the inner sheath. Retraction of the outer sheath (60) may only be accomplished by a conscious act of the surgeon and accidental retraction is substantially prevented. For this purpose, the inner sheath (40) may comprise a soft biocompatible polymeric material to encourage tearing of the slits (45a, 45b). Suitably, the inner sheath (40) may comprise longitudinally molecularly orientated, anisotropic material such as PFTE whose molecular properties permit it to be torn longitudinally along slit lines adjacent the pulling wire lumens (41a, 41b). The use of two (or more) equiangularly spaced pull wires may assist in ensuring that the retracting movement of the delivery sheath lies wholly on the longitudinal axis without rocking or twisting.

FIG. 4 displays a cross section through an introducer according to further embodiment of the present invention, in which two elongate regions of weakness that extend longitudinally along the radially exterior surface of inner sheath (40) include grooves (43a, 43b) cut into the surface of inner sheath (40) adjacent the pulling wire lumens (41a, 41b). The grooves serve to reduce the amount of material that the distal ends of the pulling wires (51a, 51b) are required to tear through. Also shown is the guide wire (10) running along the longitudinal axis of the central lumen (42) and catheter (20) extending over the length of guide wire (10).

FIG. 5 displays a cross section through a still further embodiment of the present invention, where elongate regions of weakness are formed in inner sheath (40) by providing elongate regions of frangible material (44a, 44b). These portions of frangible material are formed adjacent the pulling wire lumens (41a, 41b) so that distal tips of the pulling wires (51a, 51b) need only tear through the frangible material. The portions of frangible material (44a, 44b) are suitably manufactured in a biocompatible polymer with less resistance to tearing than the material of inner sheath (40). In particular, the frangible material may have a lower shear strength and/or scratch hardness than the material of the remainder of the sheath so that there is a substantial contrast in the resistance to tearing at the boundary of the regions of frangible material (44a, 44b). Further, the regions of frangible material (44a, 44b) may consist of longitudinally molecularly orientated, anisotropic material such as PFTE whose molecular properties permit it to be torn longitudinally along slit lines adjacent the pulling wire lumens (41a, 41b), whilst the remainder of the inner sheath consists of ordinary PFTE or a similar biocompatible polymer.

FIG. 6 displays a cross sectional view through an introducer according to a still further embodiment of the present invention. This embodiment is similar to that pictured in FIG. 4 with the addition of an annular cutting member (80) which encircles the radially exterior surface of inner sheath (40). The annular member has two opposing and radially in-facing barbs (81a, 81b), which in use pierce the surface of interior sheath (40) so that proximal movement of the annular member scores slits in the surface of inner sheath (40). Thus, the annular member facilitates proximal movement of the ends of the pulling wires (51a, 51b). In this embodiment, the tips of the barbs (81a, 81b) are of sufficient depth to reach the guide wire lumens (41a, 41b), but it will be appreciated that the barbs may penetrate to various depths in other embodiments of the present invention. The outer sheath (not shown in FIG. 6) is disposed distally to the annular cutting member and urges the annular cutting member proximally by contact between their opposing end surfaces. In some embodiments the annular cutting member (80) may be formed integrally with the outer sheath, so that in effect an outer sheath is provided with two radially in-facing barbs. The point of attachment of the distal ends of the pulling wires (51a, 51b) to the outer sheath (not shown) may be distal to the locations of the barbs (81a, 81b).

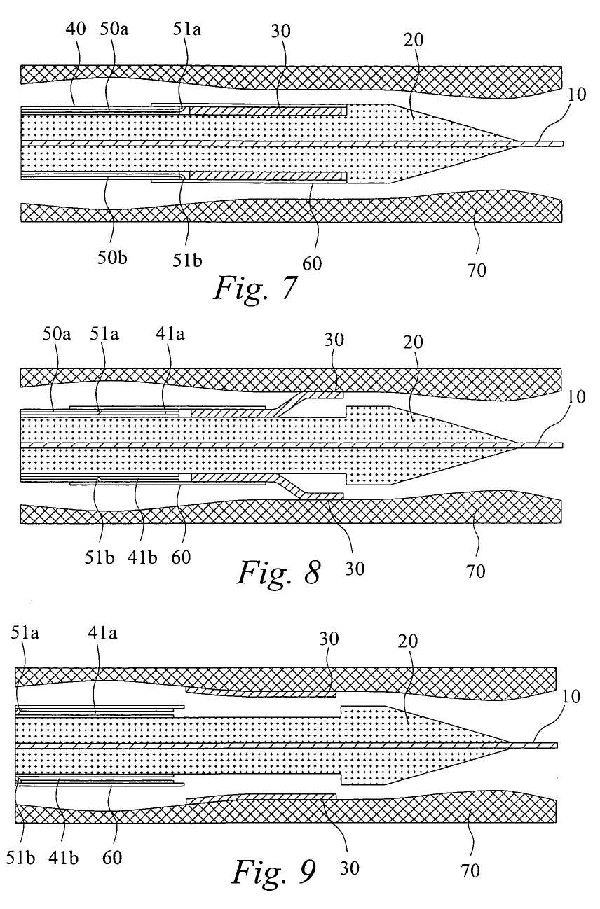

FIG. 7 displays a longitudinal cross-sectional view through the introducer of FIGS. 1, 2 and 3 when placed at a deployment location within bodily lumens (70), the section being taken through both pulling wire lumens (41a, 41b) and the central lumen (42). As may be seen from FIG. 7 the distal ends of the pulling wires (51a, 51b) are attached to the radially interior surface of the outer sheath (60).

FIG. 8 displays the introducer of FIG. 7 with the pulling wires (50a, 50b) having been pulled a short distance proximally. This causes the ends of the pulling wires (51a, 51b) to tear slits in the surface of inner sheath (40), thus allowing the retraction of outer sheath (60) over inner sheath (40). A distal portion of the self-expandable stent (30) has been unsheathed and contacts the vessel walls (70).

FIG. 9 displays the introducer of FIGS. 7 and 8 with the outer sheath (60) fully retracted. A radiopaque marker may advantageously be integrated in the distal end of the outer sheath (60) so as to enable the surgeon to visualise when the outer sheath (60) has been retracted sufficiently far to release the stent (30).

It will of course be appreciated by those skilled in the art that the foregoing description is exemplary only and that there will be a variety of ways of providing regions of weakness within the inner sheath. The use of grooves and different materials should only been seen as an example of the general principle of providing constructions, configurations or materials which provide a portion which is less resilient to tearing. For example, the use of grooves should be seen as equivalent to the use of perforation or pre-formed structural weakness of any kind.

The invention may be employed with catheter structures adapted for rapid exchange wire guide usage or with catheter structures having no wire guide lumen and adapted for insertion within a previously deployed introducer sheath.

Further, as a person skilled in the art will readily appreciate, the above description is meant as an illustration of the implementation of the principle of this invention. This description is not intended to limit the scope or application of this invention in that the invention is susceptible to modification, variation and change without departing from the spirit of the invention, as defined in the following claims. In particular, where specific combinations of features are presented in this specification, which includes the following claims and the drawings, those skilled in the art will appreciate that the features may be incorporated within the invention independently of or other disclosed and/or illustrated features.

What is claimed is:

1. Apparatus for delivering an expandable prosthesis within a body lumen, comprising a catheter structure having at its distal end a prosthesis bearing site; a delivery sheath positioned over said prosthesis bearing site so as to retain a prosthesis as the catheter structure is advanced through the body lumen, the delivery sheath being retractable to release the prosthesis; a pull wire lumen extending at least part way through the catheter structure from the proximal end of the delivery sheath; and a pull wire connected with the delivery sheath and extending through the pull wire lumen to enable remote retraction of the delivery sheath, wherein the distal end of the pull wire lumen is associated with a region of weakness in the catheter structure such that the region of weakness is torn on retraction of the delivery sheath.

2. Apparatus according to claim 1, wherein said catheter structure comprises an inner sheath, said pull wire lumen and said associated region of weakness being located in the inner sheath.

3. Apparatus according to claim 1, wherein said apparatus further comprises a barb that, on retraction of the delivery sheath, aids in tearing said region of weakness.

4. Apparatus according to claim 3, wherein said barb is formed on an annular member located around said catheter structure.

5. Apparatus according to claim 3, wherein said barb is formed on said delivery sheath.

6. Apparatus according to claim 3, comprising a plurality of said barbs spaced equiangularly about said catheter structure.

7. Apparatus according to claim 1, comprising a plurality of said pull wires spaced equiangularly about said catheter structure.

8. Apparatus according to claim 1, wherein said regions of weakness are provided at least in part by grooves formed in said catheter structure.

9. Apparatus according to claim 2, wherein said inner sheath consists substantially of a first material and said regions of weakness are provided at least in part by elongate regions of a second material.

10. Apparatus according to claim 9, wherein said first material has greater shear strength than said first material.

11. Apparatus according to claim 9, wherein said first material has greater scratch hardness than said first material.

12. Apparatus according to claim 9, wherein said second material is anisotropic.

13. Apparatus according to claim 1, further comprising a self-expandable stent disposed at said prosthesis bearing site.

14. Method for delivering an expandable prosthesis within body lumens, said method comprising the steps of positioning in a body lumen an apparatus having a longitudinal axis, said apparatus comprising a catheter structure having at its distal end a prosthesis bearing site; an expandable prosthesis located at said prosthesis bearing site; a delivery sheath positioned over said prosthesis bearing site so as to retain the prosthesis as the catheter structure is advanced through the body lumen, the delivery sheath being retractable to release the prosthesis; a pull wire lumen extending at least part way through the catheter structure from the proximal end of the delivery sheath; and a pull wire connected with the delivery sheath and extending through the pull wire lumen to enable remote retraction of the delivery sheath, wherein the distal end of the pull wire lumen is associated with a region of weakness in the catheter structure; and pulling said pull wire proximally along said axis so as to retract said delivery sheath, thus unsheathing said prosthesis, and tearing said region of weakness.

15. Method according to claim 14, wherein said expandable prosthesis is a self-expandable stent.
